SEQUENCE LISTING (15 sequences)

SEQ ID NO: 1 - DNA, 1584 bp, Clostridium phytofermentans
atgaagaagagaagaagctggttattgccattggcaggagttgttgttgcgttagttactttttttaatgcaagtccgaatgttgtagcaagatatctgattatagctaccatcgttatcatattgataggaatcgcggcagtgattgttggcttgctgcaaggcagtggttcggttgacaatggagagagttatgtaaaagatgaatttcaaaacacagcacgaaagaaattggagggtatttctgggtctattcataaattggcgtcaagctttgattatatggcatcccccaagacggtactaaatacagaggacatgcagttggtactcgaggacattagtacaaacctctgtaaaaattgtaaaaaatgtggagtatgttgggaaagaaattttaatcaaagttatcaagcaacatggaatctacttgagaccgcgaaagggaaaggaaatgtaactgtagatgatatgcctgatatgctaagacttcaatgtattcaggttcctgaatttgtggaggaggcaaaccgtaatcttagtatggctcgcctaaagatggtttggcacaaccgcattgttgagagtagggaagcggtagctgggcagcttggggagattgcaagaattgtgaaagacttctctggaaacctatgcgacaccggtgaagtaatagagttaaaacgaagaaaaataaatcaaaaacttcgtgtacatagaattaaagtgcagcgggtcttgatgtttgaacgggaaaatcgaggaatggagctgcatttaagagcaagatgcaaaaacggcaggtgcctgacaacgaaggaagcagcgatcttaattgggaatgctttagaaaggaggtttgtgcctagagaggattcaaggaatgtcattgggcgagaatacgatgattatgtcttttgtgaagatgctaattttaaggtattaacaggtgtatccagagcgtcaaagaaaaaaggtgaattaaacggagataatttttcttttctctatccagatagccaggatgttgtaatgatgctatcagatggtatggggagtggaagcgaagcttatgaagaaagtgaaatggtaattgaattattggagcagttcttagaagcagggtttcgggaagaacccgcaattaagcttatcaactctgtccttgttttgcgcaccgaaaattgcatgtcttccacagttgatttatgtgtcgttaacttatgtgcggggacatgcgaatttgtaaaaatcggagcagccaccacttttattaagagggatcattttgttgagaccataagttcgaacagtatgcctgcaggaattttaaatcgagttgactatgatactaagagcaaaaagctttatgatggtgattatgttatcatggtatcagatggggtgattgattgtgttgaagaggaggacaaagaagggtatttcataaattttataaaaaatattccttttaagtcaccacaggaaattgcaaatgcaattttatctgctgcgctagaaaagcatggttatgtgccagcagatgatatgactgtcttagttactggtatttggaaaaagtaa

SEQ ID NO: 2 - DNA, 882 bp, Clostridium phytofermentans
ttgcaactcgaagtatatatcgatgtactctttattgttaactttgttatggatctagttttattaatcattgtgaaaagattgcgccgccaggaaggaaaactatatcgattagttcttggttctattgctggtgccgcattatcgtgtttggtcaatatatattttatgtcagatatttttttatatctgctcataggttatggattgactggctttcttatgactatgattacttttggagtccaaaataagcgtgcatttataagtaattatcttgtattactaataaccacctttgttcttggaggaatggtgaattcactttatctaaatacccaaacaagatattacttaaatctcatgtatcaagagttattacaaggagagaagaggactgctacgattgtcatcatcactatcattactttgttgctttttagcttcgtctgtataatatggaggcaaaatagaaagaaggaagaagagttgtactcagtagagctctacatagaggatgagcctattttgtgcaagggattaatggatacagggaactcgttaagagatccggtttccggaaaaccagtaatagtcgtggatgaaaaactattagagaaagaaatgaatcaattaaaggaattacatccaaatcgtattcgcgttattccgtatagttctgttggtaaacataacggtcttttatttggaatccgattgaagaagattattataagtaattcaaatgactgtatctgtaatcatgaagttgtcgctgctttgtcaaatcaaggatttgcaaatcgggaggcctatcaggtattgttacatacagatcttttggggattataggtggacaggaaagtcaaatgggaaaatga

SEQ ID NO: 3 - DNA, 783 bp, Clostridium phytofermentans
atggctctttataaggtagaaatttgtggagtaaatacatcgaaattgccgttgttaaagggtgaagaaaaggatgctttatttgaaagaatcaagcaaggtgataaggaagctagagagctgtatatcaaaggaaacctacgattggtgctaagcatcatacaacgattttcaaatagtaatgaaaatgttgacgatttgtttcaaattggttgtattggtttaatgaaagcaatcgataattttgacatcacgcaaggtgtaaagttttcaacctatgcagtaccaatgataattggtgagatacgtcgttatttaagagataataatgcgatccgtgtatcccgttctttaagggatactgcttataaagcaatttatgcgaaagaaatgcttcttaagaaaaatgataaagaaccaacggtttgtgagattgcaaatgaagttggaattagtcaggaagatattgtggcagcacttgatgcaatccagagcccagtatctttgtatgagcctgtctattcagagggtggagacactttatatattatggaccaggtaagtgacaaaaagaataaagaagaaaactgggtggaagaaatctcattaaaggaagcgatggcgagattatctccaagagaaaataatattattaatcttcgtttctttcaaggaaagactcagatggaagtagctgaagaaattcaaatttcacaagcacaagttagtagacttgaaaaaaatgcattaaaaaatatgagaaactatcttatggataaataa

SEQ ID NO: 4 - DNA, 21 bp, Clostridium phytofermentans
cccttctttgtcctcctcttc

SEQ ID NO: 5 - DNA, 21 bp, Clostridium phytofermentans
cttctcctcctgtttcttccc

SEQ ID NO: 6 - DNA, 21 bp, Clostridium phytofermentans
gaagaggaggacaaagaaggg

SEQ ID NO: 7 - DNA, 21 bp, Clostridium phytofermentans
gcagtcctcttctctccttgt

SEQ ID NO: 8 - DNA, 21 bp, Clostridium phytofermentans
tgttcctctcttctcctgacg

SEQ ID NO: 9 - DNA, 21 bp, Clostridium phytofermentans
cgtcaggagaagagaggaaca

SEQ ID NO: 10 - DNA, 19 bp, Clostridium phytofermentans
gtctccaccctctgaatag

SEQ ID NO: 11 - DNA, 19 bp, Clostridium phytofermentans
gataagtctcccacctctg

SEQ ID NO: 12 - DNA, 19 bp, Clostridium phytofermentans
cagaggtgggagacttatc

SEQ ID NO: 13 - protein, 626 aa, Clostridium phytofermentans
MGKISVVIVDDNIRMLNLLEEVLKNDNDVEVIGRAENGLEALEVIKDKNPDVVLLDLIMPKLKISVIDDNNLLNDVGAGEALIKPDVLDIMPLMESRKISVLIADDNKEFCNILNDYLLNQSDMIVVGIAKDGVEALKLIENKKPDLVVLDIIMPRLDGLGVMEKIKKSSEFKKAPSFIVITAIGQERVTENAFELGASYYILKPFDNNTVLSRIKQDGLGVEKPIVAGQTALGAYYKPFDRIDGLGVLEKLNNKDAENLPRIIVLSAVGQDKITQRAITLGADYYVVKPFDMDVFTNRIRELKADYHIKLVDNHKLNTFDNPAAYKEKNLESDVTNIIHEIGVPAHKYTNKLESTIIHIGVPAHMFNNTISNSEQKRSYQVEEKEASFAGTIANDVYSDNIGNKAVDLESEITSIIHQIGVPAHIKGYQYLRDAIMMSVDDTEMLNSITKQLYPSIAKRHKTTPSRVERAIRHAIEVAWSRGKMIKGYYLRAIMVELTKLYPSIAKTTSRVERAIRHAIEVAWSRGIKGYMYLREAITMVVNNMELLSAVTKELYPSIAKKYNTTASRVERAIRHAIEVAWSRGQVDTIDDLFGYTVSNGKGKPTNSEFVALIADKIRLEYKLRM

SEQ ID NO: 14 - protein, 64 aa, Clostridium acetobutylicum
TILFGYTNGKGKPTNSEFAIADKRLKETINKLFGYTINNGKGKPTNSEFIAMIADKLRLKNKVS

SEQ ID NO: 15 - DNA, 7 bp, Bacillus subtilis
  misc_feature 3: n = A,T,C or G
tgncgaa

(19) United States
(12) Patent Application Publication
(10) Pub. No.: US 2010/0105114 A1
(43) Pub. Date: Apr. 29, 2010

(54) Methods and Compositions for Regulating Sporulation

(75) Inventors: Jeffrey Blanchard, Leverett, MA (US); Susan Leschine, Leverett, MA (US); Elsa Petit, Northampton, MA (US); John Fabel, Amherst, MA (US)

Correspondence Address: FISH & RICHARDSON PC, P.O. BOX 1022, MINNEAPOLIS, MN, 55440-1022, US

(73) Assignee: University of Massachusetts

(21) Appl. No.: 12/483,118

(22) Filed: Jun. 11, 2009

Related U.S. Application Data
(60) Provisional application No. 61/060,620, filed on Jun. 11, 2008.

(51) Int. Cl. C12P 7/62; C12P 1/04; C12P 7/06; C12P 7/04; C12N 1/21; C12N 15/74

(52) U.S. Cl. 435/135; 435/170; 435/161; 435/157; 435/252.3; 435/471; 435/476

(57) ABSTRACT

Methods and compositions are provided for producing fuel utilizing various strains of Clostridium phytofermentans with reduced sporulation activity. In some embodiments, the activity of a gene associated with sporulation is reduced. In some embodiments, a fuel producing strain of C. phytofermentans with reduced sporulation activity is provided.

### CROSS-REFERENCE TO RELATED APPLICATIONS

This application claims the benefit of priority from U.S. Provisional Patent Application No. 61/060,620, filed on Jun. 11, 2008, the contents of which are incorporated herein by reference in their entirety.

### FIELD OF THE INVENTION

Methods are provided for producing ethanol and other chemicals utilizing various strains of Clostridium phytofermentans modified to exhibit altered sporulation activity.

### BACKGROUND

There is an interest in developing methods of producing usable energy from renewable and sustainable biomass resources. Energy in the form of carbohydrates can be found in waste biomass, and in dedicated energy crops, such as grain, for example, corn or wheat, or grasses such as switchgrass.

A current challenge is to develop viable and economical strategies for the conversion of carbohydrates into usable energy forms. Strategies for deriving useful energy from carbohydrates include the production of ethanol and other alcohols, conversion of carbohydrates into hydrogen, and direct conversion of carbohydrates into electrical energy through fuel cells. Examples of strategies to derive ethanol from biomass have been described (DiPardo, Journal of Outlook for Biomass Ethanol Production and Demand (EIA Forecasts), 2002; Sheehan, Biotechnology Progress, 15:8179, 1999; Martin, Enzyme Microbes Technology, 31:274, 2002; Greer, BioCycle, 61-65, April 2005; Lynd, Microbiology and Molecular Biology Reviews, 66:3, 506-577, 2002; and Lynd et al., Current Opinion in Biotechnology, 16:577-583, 2005).

Leschine and Warwick have reported that an isolated strain of an anaerobic bacterium, C. phytofermentans (ISDg^{T}; American Type Culture Collection 700394^{T}), alone or in combination with one or more other microbes, can ferment cellulosic biomass material into a combustible biofuel, such as ethanol, propanol and/or hydrogen (U.S. Patent Application No. 2007/0178569; Warnick et. al., Int. J. Syst. Evol. Microbiol. (2002), 52 1155-1160; both references hereby incorporated expressly by reference in their entireties).

### SUMMARY

The inventions described herein are based, at least in part, on the discovery that new modified strains of C. phytofermentans can be provided, wherein the new strains have altered (e.g., reduced) activity in at least one gene involved in sporulation in comparison to wild type. The new strains can be incubated in a culture medium until the new strains produce ethanol. In further embodiments, ethanol (e.g., enhanced levels of ethanol) can be obtained from the culture medium and purified. In even further embodiments, propanol, proprionate, acetate, lactate, formate, and/or hydrogen can be obtained from the culture medium.

In additional embodiments, the gene or genes with altered activity are upregulated by Spo0A. In further embodiments, the gene(s) can be selected from the group consisting of SpoIIAA, SpoIIAB, SigF, SpoIIE, SpoIIGA, SigG, and SigE. In certain embodiments, the gene can be SpoIIE.

In additional embodiments, a strain of C. phytofermentans with altered (e.g., reduced) activity of a gene or genes associated with sporulation is obtained by disrupting the gene(s). In certain embodiments, the gene(s) are disrupted by providing an inhibitory nucleic acid (e.g., antisense oligonucleotide or ribozyme) to a C. phytofermentans cell. In further embodiments, the inhibitory nucleic acid is provided by expression from a plasmid. In certain embodiments, the gene(s) are disrupted by deleting the gene(s). In further embodiments, a portion of the gene(s) is deleted. In certain embodiments, the gene(s) are disrupted by mutating the gene(s). In certain embodiments, the gene(s) are disrupted by disrupting a promoter or expression regulatory sequence element (e.g., by deletion or mutation).

In additional embodiments, the culture medium includes a biomass material. In further embodiments, the biomass material includes hydrolyzed plant polysaccharides. In certain embodiments, the biomass material includes pectin.

In another aspect, ethanol producing strains of C. phytofermentans are provided having altered (e.g., reduced) sporulation activity. In some embodiments, a strain has at least one gene modified to provide altered sporulation activity. In further embodiments, the gene is a gene that is upregulated by Spo0A. In additional embodiments, the gene or genes modified are selected from the group consisting of SpoIIAA, SpoIIAB, SigF, SpoIIE, SpoIIGA, SigG, and SigE. In another embodiment, a gene that is modified to provide altered sporulation activity is SpoIIE.

In additional embodiments, a C. phytofermentans cell has at least one gene involved in sporulation that is disrupted. In some embodiments, a cell has at least one sporulation gene that is disrupted by an antisense oligonucleotide. In further embodiments, an oligonucleotide used to disrupt at least one sporulation gene is expressed from a plasmid or mobilizable vector. In other embodiments, a gene involved in sporulation activity is partially or completed deleted. In certain other embodiments, a gene involved in sporulation activity is mutated (e.g., by a point mutation or codon substitution).

C. phytofermentans provides several advantages relative to other biofuels-related organisms. For example, C. phytofermentans: (i) can saccharify and ferment to ethanol all major carbohydrate components of plant biomass; (ii) has a broad plant feedstock range; (iii) can ferment polysaccharides (e.g., cellulose, xylan), five and six carbon sugars (e.g., glucose, xylose or arabinose) and oxy or deoxy sugars (e.g. galactose or fucose); (iv) is genetically far removed from other biofuel-related microorganisms, including other clostridia; (v) has a greater abundance and diversity of glycoside hydrolases relative to other biofuels-related clostridia; (vi) has a greater number of sugar transport systems than other bio-fuels related clostridia; (vii) has assembled many biofuel-related activities through horizontal gene transfer; (viii) lacks the cellulosome and the scaffolding system found in other biofuels-related microorganisms; (ix) uses novel pathways to produce ethanol; (x) has a high native molar product ratio of ethanol to other products. Further, the current native biofuel capabilities of C. phytofermentans can be readily improved upon, and genetic components from C. phytofermentans can be of use in other biofuels-related microbes.

As utilized in accordance with the embodiments provided herein, the following terms, unless otherwise indicated, shall be understood to have the following meanings:

“Fuels” is used herein to refer to solid, liquid, or gaseous substances that can be combusted to produce energy, including, but not limited to hydrocarbons; hydrogen; methane; hydroxy compounds such as alcohols, for example, ethanol, butanol, propanol, methanol. The altered microorganisms described herein can also produce various organic compounds such as carbonyl compounds such as aldehydes and ketones, for example, acetone, formaldehyde, 1-propanal; organic acids; derivatives of organic acids such as esters, for example, wax esters, glycerides; and other functional compounds including, but not limited to, 1,2-propanediol, 1,3-propanediol, lactic acid, formic acid, acetic acid, succinic acid, pyruvic acid. Non-limiting examples of organic solvents and fuels include ethanol, butanol, propanol, n-propanol, isopropanol, n-butanol, or mixtures thereof; methane, and hydrogen; organic acids, such as, formic acid, lactic acid, succinic acid, pyruvic acid and acetic acid; and salts including formate, lactate, succinate, pyruvate, and acetate.

A gene “associated with” sporulation is a gene that takes part in sporulation either by promoting sporulation or by inhibiting sporulation. A gene associated with sporulation can be identified using the methods described herein, for example, by sequence identity to genes in other organisms known to be involved in sporulation, by microarray analysis of expression patterns prior and during sporulation in C. phytofermentans, by analysis of genetic pathways, and by more methods known in the art.

The “activity” of a gene refers to the level of expression and/or action of a gene. Level of expression and/or action of a gene can be measured by a variety of means including measuring levels of mRNA, protein, effect of the gene in assays, and additional methods known in the art. In some embodiments, altered activity can refer to increased or enhanced activity, or decreased or reduced activity. For example, decreased activity or reduced activity can refer to lower levels of activity of a modified gene in comparison to a wild type gene and/or unmodified gene. Increased activity can refer to greater levels of activity of a modified gene in comparison to a wild type gene and/or unmodified gene.

A first gene upregulated by a second gene is a first gene whose levels of activity are increased by the second gene.

Disruption of a gene refers to one or more modifications made to a gene, such as, for example, mutations, insertions, and deletions. For example, disruption of a gene can refer to a modification of gene activity.

Biomass refers to a biological material that can be converted into a fuel, chemical or other product. One exemplary source of biomass is plant matter. Plant matter is, for example, woody plant matter, non-woody plant matter, macroalgae matter, microalgae matter, cellulosic material, lignocellulosic material, hemicellulosic material, carbohydrates, pectin, starch, inulin, fructans, glucans, corn, sugar cane, grasses, switchgrass, bamboo, and material derived from these. Plant matter can be further described by reference to the chemical species present, such as proteins, polysaccharides, and oils. Polysaccharides include polymers of various monosaccharides and derivatives of monosaccharides including glucose, fructose, lactose, galacturonic acid, rhamnose, etc. Pectin can include pomace (e.g., from apples, pears, or other fruit), fruit processing waster, polygalacturonic acid, polysaccharides comprising D-galacturonic acid moieties (esterified with alcohols or not and/or at least a portion of which are esterified with methanol), polysaccharides comprising (1-4)-linked D-galacturonic acid units, and polysaccharides comprising (1-4)-linked D galacturonic acid units that also include regions of (1-2)-linked L-rhamnose.

Plant matter also includes agricultural waste byproducts or side streams such as pomace, corn steep liquor, corn steep solids, distillers grains, peels, pits, fermentation waste, straw, lumber, garbage and food leftovers. These materials can come from farms, forestry, industrial sources, households, etc. Another non-limiting example of biomass is animal matter, including, for example milk, meat, fat, animal processing waste, sewage, and animal waste.

“Nucleotide” refers to a phosphate ester of a nucleoside, as a monomer unit or within a nucleic acid. “Nucleotide 5′-triphosphate” refers to a nucleotide with a triphosphate ester group at the 5′ position, and is sometimes denoted as “NTP”, or “dNTP” and “ddNTP” to particularly point out the structural features of the ribose sugar. The triphosphate ester group can include sulfur substitutions for the various oxygens, e.g., α-thio-nucleotide 5′-triphosphates. For a review of nucleic acid chemistry, see: Shabarova, Z. and Bogdanov, A. Advanced Organic Chemistry of Nucleic Acids, VCH, New York, 1994.

The terms “nucleic acid” and “nucleic acid molecule” refer to natural nucleic acid sequences such as DNA (deoxyribonucleic acid) and RNA (ribonucleic acid), artificial nucleic acids, analogs thereof, or combinations thereof.

As used herein, the terms “polynucleotide” and “oligonucleotide” are used interchangeably and mean single-stranded and double-stranded polymers of nucleotide monomers (nucleic acids), including, but not limited to, 2′-deoxyribonucleotides (nucleic acid) and ribonucleotides (RNA) linked by internucleotide phosphodiester bond linkages, e.g. 3′-5′ and 2′-5′, inverted linkages, for example, 5′-5′, branched structures, or analog nucleic acids. Polynucleotides have associated counter ions, such as H+, NH4+, trialkylammonium, Mg2+, Na+ and the like. A polynucleotide can be composed entirely of deoxyribonucleotides, entirely of ribonucleotides, or chimeric mixtures thereof. Polynucleotides can be comprised of nucleobase and sugar analogs. Polynucleotides typically range in size from a few monomeric units, for example, 5-40 when they are more commonly frequently referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine, “C” denotes deoxycytidine, “G” denotes deoxyguanosine, and “T” denotes thymidine

The term “plasmid” refers to a circular nucleic acid vector. Generally, plasmids contain an origin of replication that allows many copies of the plasmid to be produced in a prokaryotic or eukaryotic cell, without integration of the plasmid into the host cell DNA.

The term “construct” as used herein refers to a recombinant nucleotide sequence, generally a recombinant nucleic acid molecule, that has been generated for the purpose of the expression of a specific nucleotide sequence(s), or is to be used in the construction of other recombinant nucleotide sequences. In general, “construct” is used herein to refer to a recombinant nucleic acid molecule.

By “expression vector” is meant a vector that permits the expression of a polynucleotide inside a cell. Expression of a polynucleotide includes transcriptional and/or post-transcriptional events. An “expression construct” is an expression vector into which a nucleotide sequence of interest has been inserted in a manner so as to be positioned to be operably linked to the expression sequences present in the expression vector.

An “operon” refers to a set of polynucleotide elements that produce a messenger RNA (mRNA). Typically, the operon includes a promoter and one or more structural genes. Typically, an operon contains one or more structural genes which are transcribed into one polycistronic mRNA: a single mRNA molecule that encodes more than one protein. In some embodiments, an operon may also include an operator that regulates the activity of the structural genes of the operon.

The term “host cell” as used herein refers to a cell that is to be transformed using the methods and compositions of the invention. In general, host cell as used herein means a microorganism cell into which a nucleic acid of interest is introduced.

The term “transformed cell” as used herein refers to a cell into which (or into an ancestor of which) has been introduced, by means of recombinant nucleic acid techniques, a nucleic acid molecule encoding a gene product of interest, for example, RNA and/or protein.

The term “gene” as used herein refers to any and all discrete coding regions of a host genome, or regions that encode a functional RNA only (e.g., tRNA, rRNA, regulatory RNAs such as ribozymes) and includes associated non-coding regions and regulatory regions. The term “gene” includes within its scope open reading frames encoding specific polypeptides, introns, and adjacent 5′ and 3′ non-coding nucleotide sequences involved in the regulation of expression. In this regard, a gene may further comprise control signals such as promoters, enhancers, and/or termination signals that are naturally associated with a given gene, or heterologous control signals. A gene sequence may be cDNA or genomic nucleic acid or a fragment thereof. A gene may be introduced into an appropriate vector for extrachromosomal maintenance or for integration into the host.

The term “promoter” as used herein refers to a minimal nucleic acid sequence sufficient to direct transcription of a nucleic acid sequence to which it is operably linked. The term “inducible promoter” as used herein refers to a promoter that is transcriptionally active when bound to a transcriptional activator, which in turn is activated under a specific condition(s), e.g., in the presence of a particular chemical signal or combination of chemical signals that affect binding of the transcriptional activator to the inducible promoter and/or affect function of the transcriptional activator itself.

The terms “operator,” “control sequences,” or “regulatory sequence,” as used herein refer to nucleic acid sequences that regulate the expression of an operably linked coding sequence in a particular host organism. The control sequences that are suitable for prokaryotes, for example, include a promoter, optionally an operator sequence, and a ribosome binding site.

By “operably connected” or “operably linked” and the like is meant a linkage of polynucleotide elements in a functional relationship. A nucleic acid sequence is “operably linked” when it is placed into a functional relationship with another nucleic acid sequence. For instance, a promoter or enhancer is operably linked to a coding sequence if it affects the transcription of the coding sequence. In some embodiments, operably linked means that the nucleic acid sequences being linked are typically contiguous and, where necessary to join two protein coding regions, contiguous and in reading frame. A coding sequence is “operably linked to” another coding sequence when RNA polymerase will transcribe the two coding sequences into a single mRNA, which is then translated into a single polypeptide having amino acids derived from both coding sequences. The coding sequences need not be contiguous to one another so long as the expressed sequences are ultimately processed to produce the desired protein.

“Operably connecting” a promoter to a transcribable polynucleotide means placing the transcribable polynucleotide under the regulatory control of a promoter, which then controls the transcription and optionally translation of that polynucleotide. In the construction of heterologous promoter/structural gene combinations, it is typical to position a promoter or variant thereof at a distance from the transcription start site of the transcribable polynucleotide, which is approximately the same as the distance between that promoter and the gene it controls in its natural setting; namely, the gene from which the promoter is derived. As is known in the art, some variation in this distance can be accommodated without loss of function. Similarly, the typical positioning of a regulatory sequence element such as an operator, enhancer, with respect to a transcribable polynucleotide to be placed under its control is defined by the positioning of the element in its natural setting; namely, the genes from which it is derived.

As used herein, the term “isolated” is intended to mean that the bacteria have been separated from an environment in which they naturally reside, and includes strains and other bacteria derived from such isolated bacteria.

“Culturing” signifies incubating a cell or organism under conditions wherein the cell or organism can carry out some, if not all, biological processes. For example, a cell that is cultured may be growing or reproducing, or it may be non-viable, but still capable of carrying out biological and/or biochemical processes such as replication, transcription, translation, etc.

“Recombinant” refers to polynucleotides synthesized or otherwise manipulated in vitro (“recombinant polynucleotides”) and to methods of using recombinant polynucleotides to produce gene products encoded by those polynucleotides in cells or other biological systems. For example, a cloned polynucleotide may be inserted into a suitable expression vector, such as a bacterial plasmid, and the plasmid can be used to transform a suitable host cell. A host cell that comprises the recombinant polynucleotide is referred to as a “recombinant host cell” or a “recombinant bacterium.” The gene is then expressed in the recombinant host cell to produce, e.g., a “recombinant protein.” In addition, a recombinant polynucleotide may serve a non-coding function, for example, promoter, origin of replication, or ribosome-binding site.

The term “homologous recombination” refers to the process of recombination between two nucleic acid molecules based on nucleic acid sequence similarity. The term embraces both reciprocal and nonreciprocal recombination, wherein nonreciprocal recombination can be also referred to as gene conversion. In addition, the recombination can be the result of equivalent or non-equivalent cross-over events. Equivalent crossing over occurs between two equivalent sequences or chromosome regions, whereas nonequivalent crossing over occurs between identical or substantially identical segments of nonequivalent sequences or chromosome regions. Unequal crossing over typically results in gene duplications and deletions. For a description of the enzymes and mechanisms involved in homologous recombination see, Watson et al., Molecular Biology of the Gene pp 313-327, The Benjamin/Cummings Publishing Co. 4th ed. (1987).

The term “non-homologous or random integration” refers to any process by which nucleic acid is integrated into the genome that does not involve homologous recombination. It appears to be a random process in which incorporation can occur at any of a large number of genomic locations.

A “heterologous polynucleotide sequence” or a “heterologous nucleic acid” is a relative term referring to a polynucleotide that is functionally related to another polynucleotide, such as a promoter sequence, in a manner so that the two polynucleotide sequences are not arranged in the same relationship to each other as in nature. Heterologous polynucleotide sequences include, e.g., a promoter operably linked to a heterologous nucleic acid, and a polynucleotide including its native promoter that is inserted into a heterologous vector for transformation into a recombinant host cell. Heterologous polynucleotide sequences are considered “exogenous” because they are introduced to the host cell via transformation techniques. However, the heterologous polynucleotide can originate from a foreign source or from the same source. Modification of the heterologous polynucleotide sequence may occur, for example, by treating the polynucleotide with a restriction enzyme to generate a polynucleotide sequence that can be operably linked to a regulatory element. Modification can also occur by techniques such as site-directed mutagenesis.

The term “expressed endogenously” refers to polynucleotides that are native to the host cell and are naturally expressed in the host cell.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification and claims are to be understood as being modified in all instances by the term “about.” The word “about” carries the understanding that a numeral referred to may vary by up to ±10%, unless indicated otherwise in the text, and still remain within the breadth and scope of an embodiment or claim element. Accordingly, unless indicated to the contrary, in some embodiments, the numerical parameters set forth in the disclosure herein are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Section headings are used herein for organizational purposes only and are not to be construed as limiting the described subject matter. When definitions of terms in incorporated references appear to differ from the definitions provided in the present teachings, the definitions provided in the present teachings shall control. It is to be understood that both the foregoing general description and the following detailed description are exemplary and explanatory only and are not restrictive of the invention. The articles “a” and “an” are used herein to refer to one or to more than one (i.e., to at least one) of the grammatical object of the article. By way of example, “an element” means one element or more than one element.

Unless otherwise defined, scientific and technical terms used in connection with the invention described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification, for example, Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein, are those well known and commonly used in the art.

All publications, patent applications, patents, and other references mentioned herein are incorporated by reference in their entirety. When definitions of terms in incorporated references appear to differ from the definitions provided in the present disclosure, the definition provided in the present disclosure shall control. In addition, to the extent incorporated references contradict the present disclosure, the disclosure supersedes and/or takes precedence over any such contradictory material.

The following figures, description, and examples illustrate certain embodiments of the present invention in detail. Those of skill in the art will recognize that there are numerous variations and modifications that are encompassed by its scope. Accordingly, the description of certain embodiments should not be deemed to limit the scope of the present invention.

### BRIEF DESCRIPTION OF THE DRAWINGS

FIG. 1 is a representation of the domain organization of the C. phytofermentans Spo0A protein.

FIG. 2 is a representation of an alignment between protein sequences of C. phytofermentans (gi 160880629; YP_{—}001559597.1) Spo0A (SEQ ID NO:13) and C. acetobutylicum (gi 15895341; NP_{—}348690.1) Spo0A (SEQ ID NO:14).

FIG. 3 is a representation of the C. phytofermentans SpoIIE sequence, Cphy0138-SpoIIE (SEQ ID NO:1).

FIG. 4 is a representation of the C. phytofermentans SpoIIGA sequence, Cphy2470-SpoIIGA (SEQ ID NO:2).

FIG. 5 is a representation of the C. phytofermentans SigG sequence, Cphy2468-SigG (SEQ ID NO:3).

FIG. 6 is a representation of a C. phytofermentans SpoIIE antisense construct (SEQ ID NOs: 5 and 6).

FIG. 7 is a representation of a C. phytofermentans SpoIIGA antisense construct (SEQ ID NOs: 8 and 9).

FIG. 8 is a representation of a C. phytofermentans SigG antisense construct (SEQ ID NOs: 11 and 12).

FIG. 9 is a neighbor joining tree of C. phytofermentans and related taxa within the class Clostridia based on 16S rRNA gene sequences. Cluster I contains disease-causing clostridia, cluster III contains cellulolytic clostridia and cluster XIVa contains gut microbes and soil isolates. Numbers at nodes are levels of bootstrap support (percentages) based on neighbor joining analyses of 1000 resampled datasets. Only values above 50 are represented. Bacillus subtilis was used as an outgroup. C. phytofermentans is within cluster XIVa, closely related to a Glade containing rice-paddy soil isolates, but divergent from human gut microbes.

### DETAILED DESCRIPTION

Various embodiments for producing a fuel, for example, ethanol, are disclosed. Some embodiments include reducing the activity of a gene associated with sporulation in a strain of C. phytofermentans; culturing the strain under conditions suitable for organic solvent production; and purifying the solvents from the culture media.

One wild-type strain of C. phytofermentans (American Type Culture Collection 700394^{T}) is defined based on the phenotypic and genotypic characteristics of a cultured strain, ISDg^{T} (Warnick et al., International Journal of Systematic and Evolutionary Microbiology, 52:1155-60, 2002). The invention generally relates to systems, methods, and compositions for producing fuels and/or other useful organic products (solvents) involving strain ISDg^{T} and/or any other strain of the species C. phytofermentans, which may be derived from strain ISDg^{T} or separately isolated. The species can be defined using standard taxonomic considerations (Stackebrandt and Goebel, International Journal of Systematic Bacteriology, 44:846-9, 1994). Strains with 16S rRNA sequence homology values of 97% and higher as compared to the type strain (ISDg^{T}) are considered strains of C. phytofermentans, unless they are shown to have DNA re-association values of less than 70%.

Considerable evidence exists to indicate that microbes that have 70% or greater DNA re-association values also have at least 96% DNA sequence identity and share phenotypic traits defining a species. Analyses of the genome sequence of C. phytofermentans strain ISDg^{T} indicate the presence of large numbers of genes and genetic loci that are likely to be involved in mechanisms and pathways for plant polysaccharide fermentation, giving rise to the unusual fermentation properties of this microbe. Based on the above-mentioned taxonomic considerations, all strains of the species C. phytofermentans would also possess all, or nearly all, of these fermentation properties. C. phytofermentans strains can be natural isolates, or genetically modified strains.

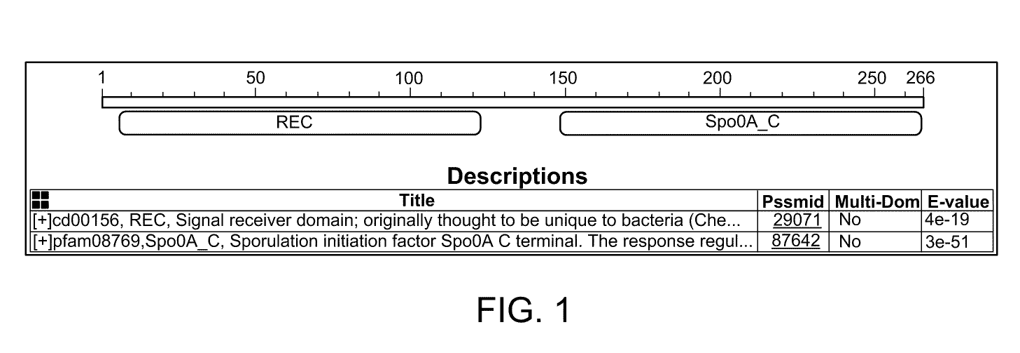

C. phytofermentans is a member of clostridia cluster XIVa. Within cluster XIVa, C. phytofermentans is mostly closely related to uncultured bacteria from anoxic rice paddy soil (FIG. 1) of the sequences deposited in Genbank. Cluster XIVa also includes human commensals that are being sequenced as part of the International Human Microbiome Consortium. C. phytofermentans is the first complete genome from this group and thus an important point of reference for comparative genomic analyses. C. phytofermentans is distantly related to clostridia cluster I, containing some pathogens and solventogenic clostridia, and cluster III, containing cellulolytic clostridia (FIG. 1). The phylogenetic analyses demonstrate that C. phytofermentans is evolutionarily related to plant debris-associated soil microbes and is distinct from other bacteria with sequenced genomes that are of interest for biofuel production.

Other embodiments include providing a recombinant solvent producing strain of C. phytofermentans. In some embodiments, one or more genes associated with sporulation have reduced activity in comparison to wild-type, wherein gene activity is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, activity is substantially or entirely eliminated. In the context of reduced activity the term “substantially” means that activity is reduced sufficiently to measurably inhibit sporulation.

In some embodiments, recombinant strains of C. phytofermentans are engineered to produce inhibitory nucleic acids (e.g., antisense nucleic acids or ribozymes) to inhibit genes associated with sporulation. For example, in some embodiments, recombinant strains of C. phytofermentans are provided with mutations in genes associated with sporulation sufficient to reduce activity of those genes. In some embodiments, mutations can be changes in the regulatory regions (e.g., promoters), premature stop codons, frame shift mutations, large insertions or deletions, or point mutations of invariant residues. In some embodiments, the mutation is a so-called “knock-out.” Other methods of reducing the activity of genes associated with sporulation can also be used.

In certain embodiments, genes associated with sporulation in C. phytofermentans can include genes upregulated by the Stage 0 Sporulation Protein A gene (Spo0A) gene. In some embodiments, a C. phytofermentans gene upregulated by Spo0A can include a gene of the SpoIIA operon, or the SpoIIG operon. Genes of the SpoIIA operon include the anti-anti-sigma factor SpoIIAA gene (Cphy0476), the anti-sigma factor SpoIIAB gene (Cphy0477), the early forespore-specific gene SigF (Cphy0478)), and SpoIIE (Cphy0138). Genes of the SpoIIG operon can include SpoIIGA (Cphy2470), SigG (Cphy2468), and the mother-cell-specific sigma factor SigE (Cphy2469).

Further embodiments include methods and compositions wherein the activity of a gene associated with sporulation is increased in comparison to wild type. In some embodiments, the activity of the Spo0A gene is increased. In other embodiments, gene activity is increased by overexpression of a gene in a cell.

Described below are embodiments to identify and isolate genes associated with sporulation in C. phytofermentans. Further embodiments include methods to reduce gene activity of genes associated with sporulation in C. phytofermentans. Other embodiments include reducing gene activity by providing a C. phytofermentans cell with a mutation. Further embodiments include reducing gene activity by providing a C. phytofermentans cell with an antisense oligonucleotide.

Identifying C. phytofermentans Sporulation Genes

Genes associated with sporulation in C. phytofermentans can be identified by a variety of methods. In some embodiments, genes associated with sporulation can be sporulation factors. In some embodiments, genes can include coding sequences, non-coding sequences, regulatory sequences (e.g., promoters), intergenic sequences, operons and clusters of genes. Methods to identify genes associated with sporulation in C. phytofermentans can include genomic or microarray analyses.

In some embodiments, a gene in C. phytofermentans can be identified by the gene's similarity to another sequence. Similarity can be determined between polynucleotide sequences or polypeptide sequences. In some embodiments, another sequence can be a sequence associated with sporulation in another organism.

In B. subtilis, differentiation into endospores involves more than 125 genes. Transcription of genes associated with sporulation is temporally and spatially controlled by at least six RNA polymerase sigma factors (σ^{A}, σ^{H}, σ^{F}, σ^{E}, σ^{G}, and σ^{K}), and at least four DNA binding proteins (Spo0A, AbrB, Hpr, and Sin) (Stragier, P., and R. Losick. 1996. Molecular genetics of sporulation in Bacillus subtilis. Annu Rev. Genet. 30:297-341). Spo0A in B. subtilis controls the initiation of sporulation, the development of competence for DNA uptake, and many other stationary-phase-associated processes. Spo0A may be phosphorylated in response to environmental, cell cycle, and metabolic signals, thus becoming activated. Once activated, Spo0A is able to activate or repress transcription at the promoters of genes that it controls (Hoch, J. A. 1993. spo0 genes, the phosphorelay, and the initiation of sporulation, p. 747-755 In A. L. Sonenshein, J. A. Hoch, and R. Losick (ed.), Bacillus subtilis and other gram-positive bacteria: biochemistry, physiology, and molecular genetics. American Society for Microbiology, Washington, D.C.). The consensus DNA binding site for phosphorylated Spo0A in B. subtilis is a 7-bp sequence (5′-TGNCGAA-3′; SEQ ID NO:15) called a 0A box.

In some embodiments, nucleotide or amino acid sequences can be analyzed using a computer algorithm or software program. In related embodiments, sequence analysis software can be commercially available or independently developed. Examples of sequence analysis software includes the GCG suite of programs (Wisconsin Package Version 9.0, Genetics Computer Group (GCG), Madison, Wis.), BLASTP, BLASTN, BLASTX (Altschul et al., J. Mol. Biol. 215:403-410 (1990), and DNASTAR (DNASTAR, Inc. 1228 S. Park St. Madison, Wis. 53715 USA), and the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Publisher: Plenum, New York, N.Y.). Typically, the default values of a program can be used, for example, a set of values or parameters originally load with the software when first initialized.

In some embodiments, the percent sequence identity can be a relationship between two or more polypeptide sequences or two or more polynucleotide sequences, as determined by comparing the sequences. In further embodiments, identity of sequences can be the degree of sequence relatedness between polypeptide or polynucleotide sequences, as the case may be, as determined by the match between strings of such sequences. Typically, sequence identity and sequence similarity can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, NY (1991).

Methods to determine sequence identity can be designed to give the best match between the sequences tested. Some methods to determine sequence identity and sequence similarity are codified in publicly available computer programs. Sequence alignments and percent identity calculations can be performed using the Megalign program of the LASERGENE bioinformatics computing suite (DNASTAR Inc., Madison, Wis.). Multiple alignment of the sequences can be performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5.

In further embodiments, microarray analysis can be implemented to identify genes associated with sporulation. Methods to implement microarray analyses to examine transcriptional programs are well known in the art. In some embodiments, a C. phytofermentans strain with either increased or decreased activity in a gene known to be associated with sporulation can be used to identify other genes associated with sporulation. For example, a gene upstream in a cascade or pathway can be used to identify other genes downstream in the cascade or pathway. In exemplary embodiments, a C. phytofermentans strain with reduced activity in Spo0A can be used to identify other genes associated with sporulation and solventogenesis using microarray analysis. In alternative embodiments, a C. phytofermentans strain with increased activity in the Spo0A can be used to identify other genes associated with sporulation and solventogenesis using microarray analysis.

In certain embodiments, a associated with sporulation in C. phytofermentans is a gene upregulated by the Spo0A gene. In some embodiments, a C. phytofermentans gene upregulated by Spo0A can include a gene of the SpoIIA operon, or the SpoIIG operon. Genes of the SpoIIA operon can include the anti-anti-sigma factor SpoIIAA gene (Cphy0476), the anti-sigma factor SpoIIAB gene (Cphy0477), the early forespore-specific gene SigF (Cphy0478)), and SpoIIE (Cphy0138). Genes of the SpoIIG operon can include SpoIIGA (Cphy2470), SigG (Cphy2468) and the mother-cell-specific sigma factor SigE (Cphy2469).

Isolating C. phytofermentans Sporulation Genes

Genes associated with sporulation in C. phytofermentans can be isolated using the sequence of an identified gene. Standard recombinant DNA and molecular cloning techniques that can be used are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et al., Current Protocols in Molecular Biology, published by Greene Publishing Assoc. and Wiley-Interscience (1987). Additionally, methods to isolate homologous or orthologous genes using sequence-dependent protocols are well known in the art. Examples of sequence-dependent protocols include, but are not limited to, methods of nucleic acid hybridization, and methods of DNA and RNA amplification as exemplified by various uses of nucleic acid amplification technologies, such as, polymerase chain reaction (PCR; Mullis et al., U.S. Pat. No. 4,683,202), ligase chain reaction (LCR; Tabor, S. et al., Proc. Acad. Sci. USA 82, 1074, (1985)) or strand displacement amplification (SDA; Walker, et al., Proc. Natl. Acad. Sci. U.S.A., 89, 392, (1992)).

In other embodiments, a gene is isolated from a C. phytofermentans DNA library by screening the library using a portion of the identified gene as a DNA hybridization probe. Examples of probes can include DNA probes labeled by methods such as, random primer DNA labeling, nick translation, or end-labeling techniques, and RNA probes produced by methods such as, in vitro transcription systems. Additionally, specific oligonucleotides can be designed and used to amplify a part of or full-length of the instant sequences. The resulting amplification products can be labeled directly during amplification reactions or labeled after amplification reactions, and used as probes to isolate full length DNA fragments under conditions of appropriate stringency.

Typically, in PCR-type amplification techniques, the primers have different sequences and are not complementary to each other. Depending on the desired test conditions, the sequences of the primers should be designed to provide for both efficient and faithful replication of the target nucleic acid. Methods of PCR primer design are common and well known in the art (Thein and Wallace, “The use of oligonucleotide as specific hybridization probes in the Diagnosis of Genetic Disorders”, in Human Genetic Diseases: A Practical Approach, K. E. Davis Ed., (1986) pp. 33-50 IRL Press, Herndon, Va.; Rychlik, W. (1993) In White, B. A. (ed.), Methods in Molecular Biology, Vol. 15, pages 31-39, PCR Protocols: Current Methods and Applications. Humania Press, Inc., Totowa, N.J.).

Generally, two short segments of an identified sequence can be used in PCR protocols to amplify longer nucleic acid fragments encoding homologous genes from DNA or RNA. The PCR can be performed on a library of cloned nucleic acid fragments wherein the sequence of one primer is derived from the identified nucleic acid sequence, and the sequence of the other primer is based upon sequences derived from a cloning vector. For example, the RACE protocol (Frohman et al., PNAS USA 85:8998 (1988)) provides a means to generate cDNAs using PCR to amplify copies of the region between a single point in the transcript and the 3′ or 5′ end. Primers oriented in the 3′ and 5′ directions can be designed from the identified sequence. Using commercially available 3′ RACE or 5′ RACE systems (BRL), specific 3′ or 5′ cDNA fragments can be isolated (Ohara et al., PNAS USA 86:5673 (1989); Loh et al., Science 243:217 (1989)).

In some embodiments, isolated nucleic acids are cloned into vectors. Typically, vectors have the ability to replicate in a host microorganism. Numerous vectors are known, for example, bacteriophage, plasmids, viruses, or hybrids thereof. Vectors can be operable as cloning vectors or expression vectors in the selected host cell. Typically, a vector comprises an isolated nucleic acid, a selectable marker, and sequences allowing autonomous replication or chromosomal integration. Further embodiments can comprise a promoter sequence driving expression of an isolated nucleic acid, an enhancer, or a termination sequence. In other embodiments, a vector can comprise sequences that allow excision of sequences subsequent to integration into chromosomal DNA of vector sequences. Examples include loxP sequences or FRT sequences, these sequences are responsive to CRE recombinase and FLP recombinase, respectively.

Reducing Activity of Sporulation Genes in C. phytofermentans

As described below, the activity of a gene associated with sporulation in C. phytofermentans can be reduced by a variety of methods. Activity of a gene associated with sporulation in C. phytofermentans can be reduced by about 5%, 10%, 15%, 20%, 25%, 30%, 35%, 40%, 45%, 50%, 55%, 60%, 65%, 70%, 75%, 80%, 85%, 90%, or 95%. In some embodiments, activity is mostly or completely eliminated. In some embodiments, activity can be measured by levels of gene expression, for example, levels of a RNA species in a cell, levels of a protein in a cell. In some embodiments, activity can be assayed by the effect of a gene product in a cell. Typically, percentage activity can be measured with respect to a control strain, for example a wild type strain of C. phytofermentans.

Certain embodiments can include methods where a sequence has been identified. For example, some embodiments include providing a C. phytofermentans cell with a specific mutation. Other embodiments include providing an antisense oligonucleotide or ribozyme to a C. phytofermentans cell. Alternatively, in some embodiments gene activity can be reduced by non-specific mutagenesis of a C. phytofermentans cell followed by screening and identifying a mutant with reduced gene activity.

With respect to embodiments that include providing a C. phytofermentans cell with a mutation in an endogenous gene with a mutation, non-limiting examples of a mutation can include changes in a regulatory region, a premature stop codon, a frame shift mutation, an insertion or deletion, or point mutation of an invariant residue. In some embodiments, a gene associated with sporulation is inactivated by a knock-out. Typically, a mutation can be introduced into an endogenous gene of C. phytofermentans by homologous recombination between targeted chromosomal DNA and a vector comprising isolated sequences. In some embodiments, a selection cassette can be excised subsequent to integration of vector sequences into chromosomal DNA. A selection cassette can comprise a selectable marker flanked by site-specific recombination sequences.

In certain embodiments, a deletion-type mutation can be made. In some embodiments, a C. phytofermentans cell can be provided with a vector comprising an isolated sequence of an endogenous gene, wherein the isolated sequence is interrupted. In further embodiments, the isolated sequence can correspond to two non-contiguous sequences of the endogenous gene. Typically, the two endogenous non-contiguous sequences flank the endogenous sequence to be deleted. Examples of sequences to be deleted can include a regulatory sequence, coding sequence, and a whole endogenous gene sequence.

In other embodiments, a replacement-type of mutation can be made. In some embodiments a C. phytofermentans cell is provided with a vector comprising an isolated sequence of an endogenous gene, further comprising a mutation. Mutations can be introduced using techniques well known in the art, for example by PCR using mismatched primers. In further embodiments, the isolated sequence further comprises a selection cassette. Examples of replacement-type mutations include insertion of a non-endogenous sequence into the endogenous gene, such as a selection cassette, and mutation of regulatory sequences.

In some embodiments, a selection cassette can be excised subsequent to integration of vector sequences into chromosomal DNA. For example, the selection cassette may be flanked by site specific recombination sites that allow specific excision. Examples of recombination sites include loxP and FRT sequences. Examples of selectable markers include genes providing antibiotic resistance, such as thiamphenicol, chloramphenicol, and macrolide-linosamide-streptogramin B. In some embodiments, the selection cassette can be excised from vector sequences integrated into chromosomal DNA by providing the cell with a site specific recombinase, for example, CRE-recombinase, or FLP recombinase. In some embodiments, an additional vector comprising a gene encoding a site-specific recombinase is transformed into a C. phytofermentans cell

Generally, C. phytofermentans strains can be grown anaerobically in Clostridial Growth Medium (CGM) at 37° C. supplemented with an appropriate antibiotic, such as 40 μg/ml erythromycin/chloramphenicol or 25 μg/mlthiamphenicol (Hartmanis and Gatenbeck. Appl. Environ. Microbiol. 47: 1277-83 (1984)). In addition, C. phytofermentans strains can be cultured in closed-cap batch fermentations of 100 ml CGM supplemented with the appropriate antibiotic 37° C. in a FORMA SCIENTIFIC™ anaerobic chamber (THERMO FORMA™, Marietta, Ohio).

In other embodiments, C. phytofermentans can be cultured according to the techniques of Hungate (Hungate, R. E. (1969). A roll tube method for cultivation of strict anaerobes. Methods Microbiol 3B, 117-132). Medium GS-2C can be used for enrichment, isolation and routine cultivation of strains of C. phytofermentans, and can be derived from GS-2 of Johnson et at (Johnson, E. A., Madia, A. & Demain, A. L. (1981). Chemically defined minimal medium for growth of the anaerobic cellulolytic thermophile Clostridium thermocellum. Appl Environ Microbiol 41, 1060-1062.). GS-2C can contain the following: 6.0 g/l ball-milled cellulose (Leschine, S. B. & Canale-Parola, E. (1983). Mesophilic cellulolytic clostridia from freshwater environments. Appl Environ Microbiol 46, 728-737.); 6.0 g/l yeast extract; 2.1 g/l urea; 2.9 g/l K₂HPO₄; 1.5 g/l KH₂PO₄; 10.0 g/l MOPS; 3.0 g/l trisodium citrate dihydrate; 2.0 g/l cysteine hydrochloride; 0.001 g/l resazurin; with the pH adjusted to 7.0. Broth cultures can be incubated in an atmosphere of O₂-free N₂ at 30° C. Cultures on plates of agar media can be incubated at room temperature in an atmosphere of N₂/CO₂/H₂ (83:10:7) in an anaerobic chamber (Coy Laboratory Products).

Typically, prior to transformation into C. phytofermentans, vectors comprising plasmid DNA can be methylated to prevent restriction by Clostridial endonucleases (Mermelstein and Papoutsakis. Appl. Environ. Microbiol. 59: 1077-1081 (1993)). In some embodiments, methylation can be accomplished by the phi3TI methyltransferase. In further embodiments, plasmid DNA can be transformed into DH10β. E. coli harboring vector pDHKM (Zhao, et al. Appl. Environ. Microbiol. 69: 2831-41 (2003)) carrying an active copy of the phi3TI methyltransferase gene.

C. phytofermentans can be transformed with vectors by a variety of methods. Methods of transformation can include electroporation and conversion of cells to protoplasts prior to transformation. In some embodiments, electrotransformation of methylated plasmids into C. phytofermentans can be carried out according to a protocol developed by Mermelstein (Mermelstein, et al. Bio/Technology 10: 190-195 (1992)). More methods can include transformation by conjugation. In other embodiments, positive transformants can be isolated on agar-solidified CGM supplemented with the appropriate antibiotic.

A mutant is identified from purified DNA using techniques well known in the art. In some embodiments, selection cassettes may be excised from integrated vector sequences using site-specific recombinases.

In some embodiments, the activity of a gene associated with sporulation is reduced by providing an antisense oligonucleotide to a C. phytofermentans cell. In some embodiments, an antisense oligonucleotide is expressed from a sequence integrated into chromosomal DNA. In other embodiments, an antisense oligonucleotide is expressed from an exogenous nucleic acid, for example a non-integrated vector.

An antisense vector can be designed according methods known in the art (e.g., Desai and Papoutsakis. Appl. Environ. Microbiol. 65: 936-45 (1999)). In one embodiment, an antisense construct comprises an antisense oligonucleotide expressed from a promoter constitutive in C. phytofermentans, for example, the phosphotransbutyrylase (ptb) promoter. The target of the antisense oligonucleotide can be selected to include sequences 5′ of the targeted gene encompassing a predicted ribosome binding site, the putative ATG start codon, and approximately 10 codons of the targeted gene. A terminator region can be selected to terminate transcription of the antisense oligonucleotide, such as the rho-independent terminator region of the naturally occurring antisense RNA targeted against the glutamine synthetase (glnA) gene of Clostridium sp. strain NCP262 (Fierro-Monti, I. P., S. J. Reid, and D. R. Woods. (1992). Differential expression of a Clostridium acetobutylicum antisense RNA: implications for regulation of glutamine synthetase. J. Bacteriol. 174:7642-7647). The antisense vector can be constructed by allowing two single-stranded oligonucleotides to anneal to each other to produce a double-stranded antisense oligonucleotide, and the double-stranded antisense oligonucleotide can then be cloned into an appropriate vector. A C. phytofermentans cell can be transformed with the antisense vector. In some embodiments, stable transformants where the antisense vector has integrated into the genome can be selected using techniques well known in the art.

In other embodiments, the activity of genes associated with sporulation in C. phytofermentans can be reduced by non-specific mutagenesis and then screening for mutants with altered (e.g., reduced) sporulation activity. Mutants with altered sporulation activity can be designated based on the stage at which the sporulation process is blocked. Mutants that do not initiate sporulation are designated as blocked at stage 0 or 1; mutants that form one or more sporulation septa are designated as blocked at stage 11 (Piggot and Coote. (1976). Genetic aspects of bacterial endospore formation. Bacteriol. Rev. 40:908-962). Random mutations can be introduced into cells by variety of methods, for example, exposure to UV radiation or a chemical agent, such as HNO₂, NH₂OH, acridine dyes, ethidium bromide (Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36, 227, (1992)).

In other embodiments, targeted deletion of genes associated with sporulation can be performed using the commercially available gene knockout systems or similar methods. The applicability of these methods to Clostridia has been demonstrated (see, e.g., Heap et al. (2007). J. Microbiol. Methods. 70:452-464; Chen et al. (2007). Plasmid. 58:182-189).

Further embodiments can include introducing random mutations using transposable elements or transposons. Transposons are genetic elements that insert randomly in DNA. Transposition methods involve the use of a transposable element in combination with a transposase enzyme. When the transposable element or transposon, is contacted with a nucleic acid fragment in the presence of the transposase, the transposable element will randomly insert into the nucleic acid fragment. The technique can be useful for random mutagenesis and for gene isolation, since the disrupted gene can be identified on the basis of the sequence of the transposable element. Kits for in vitro transposition are commercially available, for example, The Primer Island Transposition Kit, available from Perkin Elmer Applied Biosystems, Branchburg, N.J., based upon the yeast Ty1 element; The Genome Priming System, available from New England Biolabs, Beverly, Mass.; based upon the bacterial transposon Tn7; and the EZ::TN Transposon Insertion Systems, available from Epicentre Technologies, Madison, Wis., based upon the Tn5 bacterial transposable element.

In some embodiments, a C. phytofermentans cell with reduced activity of a gene associated with sporulation can be identified by screening a series of mutants. In further embodiments, the mutants can be natural mutants or mutants generated using non-specific means. Methods to screen for mutants with reduced activity in sporulation are well known in the art, for example, by visualizing the morphology of a population of C. phytofermentans, or measuring chemical markers associated with sporulation, such as genes with altered expression patterns immediately prior or during sporulation, in comparison to wild-type.

In some embodiments, the activity of genes associated with sporulation can be increased. In certain embodiments, a gene associated with sporulation can be overexpressed. In an exemplary embodiment, Spo0A gene activity is increased. In some embodiments, the Spo0A gene is cloned into a vector and coupled to a promoter constitutive in C. phytofermentans. A cell can be transformed with a vector carrying the Spo0A gene.

General Methods of Fermentation Using Modified C. phytofermentans Strains

In some embodiments, a strain of C. phytofermentans that exhibits reduced sporulation can ferment a broad spectrum of materials into fuels with high efficiency (Co-pending U.S. Patent Application No. 2007/0178569 and U.S. Provisional Patent Application No. 61/032,048, filed Feb. 28, 2008; both references hereby incorporated by reference in their entireties). In further embodiments, the strain can be recombinant. In some embodiments, the strain can have altered (e.g., reduced) sporulation activity. In other embodiments, the strain can have reduced activity in a gene associated with sporulation. In further embodiments, genes associated with sporulation can include a gene upregulated by the Spo0A gene. In some embodiments, a C. phytofermentans gene upregulated by Spo0A can include a gene of the SpoIIA operon, or the SpoIIG operon. Genes of the SpoIIA operon can include the anti-anti-sigma factor SpoIIAA gene (Cphy0476), the anti-sigma factor SpoIIAB gene (Cphy0477), the early forespore-specific gene SigF (Cphy0478)), and SpoIIE (Cphy0138). Genes of the SpoIIG operon can include SpoIIGA (Cphy2470), SigG (Cphy2468) and the mother-cell-specific sigma factor SigE (Cphy2469).

In certain embodiments, a modified strain of C. phytofermentans can ferment waste biomass into fuel, solvents, and useful compounds.

In other embodiments, recombinant strains of C. phytofermentans can be used alone or in combination with one or more other microbes. Examples of other microbes can include yeast or fungi, such as, Saccharomyces cerevisiae, Pichia stipitis, Trichoderma species, Aspergillus species; and other bacteria such as, Zymomonas mobilis, Klebsiella oxytoca, Escherichia coli, Clostridium acetobutylicum, C. aminovalericum, C. jejuense, Clostridium beijerinckii, Clostridium papyrosolvens, Clostridium cellulolyticum, Clostridium josui, Clostridium termitidis, Clostridium cellulose, Clostridium celerecrescens, Clostridium populeti, and Clostridium cellulovorans. In further embodiments, mixtures of microbes can be provided as solid mixtures, such as, freeze-dried mixtures, or as liquid dispersions of the microbes, and grown in co-culture with C. phytofermentans. Alternatively, microbes can be added sequentially to the culture medium, for example, by adding another microbe before or after addition of C. phytofermentans.

In some embodiments, fuels and organic solvents can be produced on a large scale using a modified strain of C. phytofermentans as described in U.S. Patent Application Publication No. 2007/0178569; hereby incorporated by reference in its entirety. In further embodiments, biomass material without pretreatment can be fermented with C. phytofermentans. In other embodiments, biomass material comprising high molecular weight carbohydrates can be hydrolyzed to lower molecular weight carbohydrates before fermentation with C. phytofermentans. Hydrolysis can be accomplished using chemical, enzymatic, or physical methods. Methods of hydrolysis can include the use of an acid such as sulfuric acid or hydrochloric acid; a base, such as sodium hydroxide, or lime; a hydrothermal process; an ammonia fiber explosion process; an enzyme; or any combination thereof.

In some embodiments, compounds (e.g., fuels and organic solvents) can be purified from biomass fermented with C. phytofermentans by a variety of means. In other embodiments, ethanol, propanol, proprionate, acetate, lactate, formate or hydrogen can be purified from the biomass. In certain embodiments, organic solvents are purified by distillation. In exemplary embodiments, about 96% ethanol can be distilled from the fermented mixture. In further embodiments, fuel grade ethanol, namely about 99-100% ethanol, can be obtained by azeotropic distillation of about 96% ethanol. Azeotropic distillation can be accomplished by the addition of benzene to about 96% ethanol and then re-distilling the mixture. Alternatively, about 96% ethanol can be passed through a molecular sieve to remove water.

In some embodiments of the methods and compositions described herein, recombinant strains of C. phytofermentans can increase production of fuel and other useful compounds in comparison to wild type strains of C. phytofermentans. In such embodiments, there can be an increase in production of fuel as compared to wild type, for example, an increase of more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, more than about 95%, more than about 100%. In other embodiments, an increase in production of fuel as compared to wild type can be greater than 100%.

Growth and Fuel Production

In some embodiments, a modified microorganism is incubated under conditions that depend on the specific fuel to be produced, and on the specific modifications to the genes associated with sporulation. The incubation conditions are designed to allow fermentation with minimal or no sporulation.

In some instances, the concentration of the microorganism suspended in the culture medium is from about 10⁶ to about 10⁹ cells/mL, e.g., from about 10⁷ to about 10⁸ cells/mL. In some implementations, the concentration at the start of fermentation is about 10⁷ cells/mL. Clostridium phytofermentans cells can ferment both low, e.g., 0.01 mM to about 5 mM, and high concentrations of carbohydrates, and are generally not inhibited in their action at relatively high concentrations of carbohydrates, which would have adverse effects on other organisms. The same is true for the modified microorganism described herein. For example, the concentration of the carbohydrate in the medium can be greater than 20 mM, e.g., greater than 25 mM, 30 mM, 40 mM, 50 mM, 60 mM, 75 mM, 100 mM, 150 mM, 200 mM, 250 mM, 300 mM, or even greater than 500 mM or more. In any of these embodiments, the concentration of the carbohydrate is generally less than 2,000 mM.

In some embodiments, a fermentable material can be, or can include, one or more low molecular weight carbohydrates, e.g., mixtures of different carbohydrates. The low molecular weight carbohydrate can be, e.g., a monosaccharide, a disaccharide, an oligiosaccharide, or mixtures of these. The monosaccharide can be, e.g., a triose, a tetrose, a pentose, a hexose, a heptose, a nonose, or mixtures of these. For example, the monosaccharide can be arabinose, glyceraldehyde, dihydroxyacetone, erythrose, ribose, ribulose, xylose, glucose, galactose, mannose, fucose, fructose, sedoheptulose, neuraminic acid, or mixtures of these. The disaccharide can be, e.g., sucrose, lactose, maltose, gentiobiose, or mixtures of these.

In some embodiments, the low molecular weight carbohydrate is generated by breaking down a high molecular weight polysaccharides (e.g., cellulose, xylan or other components of hemicellulose, pectin, and/or starch). This technique can be advantageously and directly applied to waste streams, e.g., waste paper (e.g., waste newsprint and waste cartons). In some instances, the breaking down is done as a separate process, and then the low molecular weight carbohydrate utilized in culturing the new recombinant microorganism described herein. In other instances, the high molecular weight carbohydrate is added directly to the medium, and is broken down into the low molecular weight carbohydrate in-situ. In some implementations, this is done chemically, e.g., by oxidation, base hydrolysis, and/or acid hydrolysis. Chemical hydrolysis has been described by Bjerre, Biotechnol. Bioeng., 49:568, 1996, and Kim et al., Biotechnol. Prog., 18:489, 2002.

Various media for growing a variety of microorganisms are known in the art. Growth media may be minimal/defined or complete/complex. Fermentable carbon sources can include any biomass material, including pretreated (e.g., by cutting, chopping, or wetting), or non-pretreated feedstock containing cellulosic, hemicellulosic, and/or lignocellulosic material. The various types of biomass include plant biomass and municipal waste biomass (residential and light commercial refuse with recyclables such as metal and glass removed).

The terms “plant biomass” and “lignocellulosic biomass” refer to any plant-derived organic matter (woody or non-woody) available for energy on a sustainable basis. Plant biomass can include, but is not limited to, agricultural crop wastes and residues such as corn stover, wheat straw, rice straw, sugar cane bagasse, and the like. Plant biomass further includes, but is not limited to, trees, woody energy crops, wood wastes and residues such as softwood forest waste, sawdust, paper and pulp industry waste streams, wood fiber, and the like. Additionally grass crops, such as switchgrass and the like have potential to be produced on a large-scale as another plant biomass source. Other types of plant biomass include yard waste (e.g., grass clippings, leaves, tree clippings, and brush) and vegetable processing waste.

“Lignocellulosic materials” include cellulose and a percentage of lignin, e.g., at least about 0.5 percent by weight to about 60 percent by weight or more lignin. These materials include plant biomass such as, but not limited to, non-woody plant biomass, cultivated crops, such as, but not limited to, grasses, for example, but not limited to, grasses, such as switchgrass, cord grass, rye grass, miscanthus, or a combination thereof, or sugar processing residues such as bagasse, or beet pulp, agricultural residues, for example, soybean stover, corn stover, rice straw, rice hulls, barley straw, corn cobs, wheat straw, canola straw, rice straw, oat straw, oat hulls, corn fiber, wood pulp fiber, sawdust, hardwood, softwood, or a combination thereof. Further, the lignocellulosic materials may include cellulosic waste material such as, but not limited to, newsprint, recycled paper, and cardboard.

In particular implementations, the lignocellulosic material is obtained from trees, such as Coniferous trees, e.g., Eastern Hemlock (Tsuga canadensis), Maidenhair Tree (Ginkgo bilboa), Pencil Cedar (Juniperus virgineana), Mountain Pine (Pinus mugo), Deodar (Cedrus deodara), Western Red Cedar (Thuja plicata), Common Yew (Taxus baccata), Colorado Spruce (Picea pungens); or Deciduous trees, e.g., Mountain Ash (Sorbus), Gum (Eucalyptus gunnii), Birch (Betula platyphylla), or Norway Maple (Acer platanoides), can be utilized. Poplar, Beech, Sugar Maple and Oak trees may also be utilized.

In some instances, the modified microorganisms can ferment lignocellulosic materials directly without the need to remove lignin. However, in certain embodiments, it is useful to remove at least some of the lignin from lignocellulosic materials before fermenting. For example, removal of the lignin from the lignocellulosic materials can make the remaining cellulosic material more porous and higher in surface area, which can, e.g., increase the rate of fermentation and ethanol yield. The lignin can be removed from lignocellulosic materials, e.g., by sulfite processes, alkaline processes, or by Kraft processes. Such process and others are described in Meister, U.S. Pat. No. 5,138,007, and Knauf et al., International Sugar Journal, 106:1263, 147-150 (2004).

These biomass, e.g., cellulosic, materials can be pretreated before being added to a culture medium. In some cases, methods of processing begin with a physical preparation of the biomass material, e.g., size reduction of raw biomass materials, such as by cutting, grinding, crushing, shearing, or chopping. In some cases, loose materials (e.g., recycled paper or switchgrass) are prepared by shearing or shredding. Screens and/or magnets can be used to remove oversized or undesirable objects such as, for example, rocks or nails from the feed stream.

In some embodiments, the biomass material to be processed is in the form of a fibrous material that includes fibers provided by shearing, cutting, or chopping a fiber source. For example, the shearing can be performed with a knife system, such as a rotary knife cutter system. As an alternative to shredding, the biomass material can be reduced in size by cutting to a desired size using a guillotine cutter.

Once the biomass material is sufficiently pretreated and added to a culture medium, additional nutrients can be, but need not always be, added to the culture medium. Such additional nutrients include nitrogen-containing compounds such as proteins, hydrolyzed proteins, ammonia, urea, nitrate, nitrite, soy, soy derivatives, casein, casein derivatives, milk powder, milk derivatives, whey, hydrolyze yeast, autolyzed yeast, corn steep liquor, corn steep solids, monosodium glutamate, and/or other fermentation nitrogen sources, vitamins, and/or mineral supplements.

In some embodiments additional culture medium components include buffers, e.g., NaHCO₃, NH₄Cl, NaH₂PO₄.H₂O, K₂HPO₄, and KH₂PO₄; electrolytes, e.g., KCl, and NaCl; growth factors; surfactants; and chelating agents. Additional growth factors can include, e.g., biotin, folic acid, pyridoxine.HCl, riboflavin, urea, yeast extracts, thymine, tryptone, adenine, cytosine, guanosine, uracil, nicotinic acid, pantothenic acid, B12 (Cyanocobalamine), p-aminobenzoic acid, and thioctic acid. Minerals can include, e.g., MgSO₄, MnSO₄.H₂O, FeSO₄.7H₂O, CaCl₂.2H₂O, CoCl₂.6H₂O, ZnCl₂, CuSO₄.5H₂O, AlK(SO₄)₂.12H₂O, H₃BO₃, Na₂MoO₄, NiCl₂.6H₂O, and NaWO₄.2H₂O. Chelating agents can include, e.g., nitrilotriacetic acid. Surfactants can include, e.g., polyethylene glycol (PEG), polypropylene glycol (PPG), copolymers of PEG and PPG, and polyvinylalcohol.

The temperature of the medium is generally maintained at less than about 45° C., e.g., less than about 42° C. (e.g., between about 34° C. and 38° C., or about 37° C.). In general, the medium is maintained at a temperature above about 5° C., e.g., above about 15° C. The pH of the medium is generally maintained below about 9.5, e.g., between about 6.0 and 9.0, or between about 8 and 8.5. Generally, during fermentation, the pH of the medium typically does not change by more than 1.5 pH units. For example, if the fermentation starts at a pH of about 7.5, it typically does not go lower than pH 6.0 at the end of the fermentation, which is within the growth range of the cells. The pH of the fermentation broth can be adjusted using neutralizing agents such as calcium carbonate or hydroxides. The selection and incorporation of any of the above fermentative methods is highly dependent on the host strain and the preferred downstream process.

In some embodiments, one or more additional lower molecular weight carbon sources can be added or be present such as glucose, sucrose, maltose, corn syrup, and lactic acid. In some embodiments, one possible form of growth media can be modified Luria-Bertani (LB) broth (with 10 g Difco tryptone, 5 g Difco yeast extract, and 5 g sodium chloride per liter). In other embodiments of the invention, cultures of constructed strains of the invention can be grown in NBS mineral salts medium and supplemented with 2% to 20% sugar (w/v) or either 5% or 10% sugar (glucose or sucrose). The microorganisms can be grown in or on NBS mineral salts medium.

Fuel production can be observed by standard methods known to those skilled in the art. In some embodiments, fermentors that include a medium that includes the recombinant microorganisms dispersed therein are configured to continuously remove a fermentation product, such as ethanol. In some embodiments, the concentration of the desired product remains substantially constant, or within about twenty five percent of an average concentration, e.g., measured after 2, 3, 4, 5, 6, or 10 hours of fermentation at an initial concentration of from about 10 mM to about 25 mM. In some embodiments, any biomass material or mixture described herein is continuously fed to the fermentors.

Clostridium phytofermentans cells adapt to relatively high concentrations of ethanol, e.g., 7 percent by weight or higher, e.g., 12.5 percent by weight. Thus, the same can be true for the transformed microorganisms described herein. These microorganisms can be grown in an ethanol rich environment prior to fermentation, e.g., 7 percent ethanol, to adapt the cells to even higher concentrations of ethanol, e.g., 20 percent. In some embodiments, the microorganisms are adapted to successively higher concentrations of ethanol, e.g., starting with 2 percent ethanol, then 5 percent ethanol, and then 10 percent ethanol.

In some embodiments, growth and production of the recombinant microorganisms disclosed herein can be performed in normal batch fermentations, fed-batch fermentations, or continuous fermentations. In some embodiments, the recombinant microorganisms are grown using batch cultures. In some embodiments, the recombinant microorganisms are grown using bioreactor fermentation. In some embodiments, the growth medium in which the recombinant microorganisms are grown is changed, thereby allowing increased levels of fuel production. The number of medium changes may vary.

There are two basic approaches to produce fuels such as ethanol or hydrogen from biomass on a large scale using the recombinant microorganisms described herein. In the first method, one first hydrolyzes, e.g., using chemical or enzymatic pretreatment, a biomass material that includes high molecular weight carbohydrates to lower molecular weight carbohydrates, and then ferments the lower molecular weight carbohydrates using the recombinant microorganisms to produce the fuel. In the second method, one ferments the biomass material itself without chemical and/or enzymatic pretreatment. For more details on large-scale production of fuels, see, e.g., U.S. Patent Application No. 2007/0178569.

### EXAMPLES

### Example 1

### Identification of Spo0A in C. phytofermentans

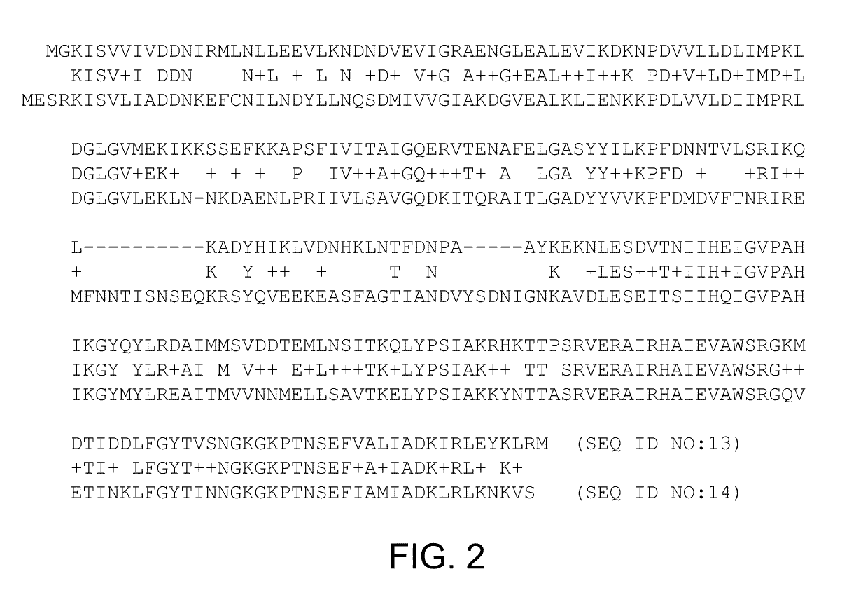

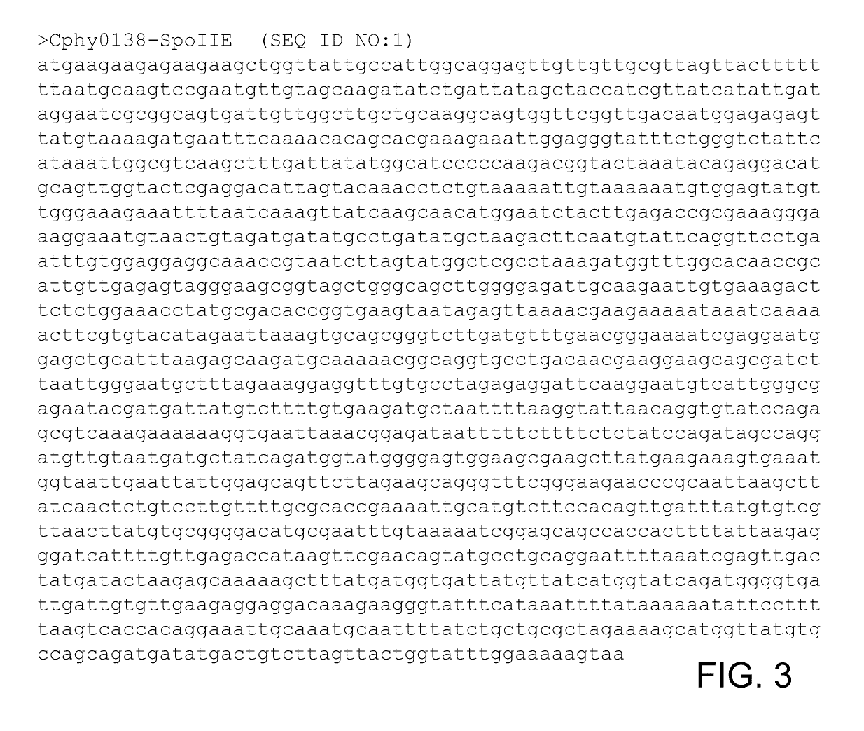

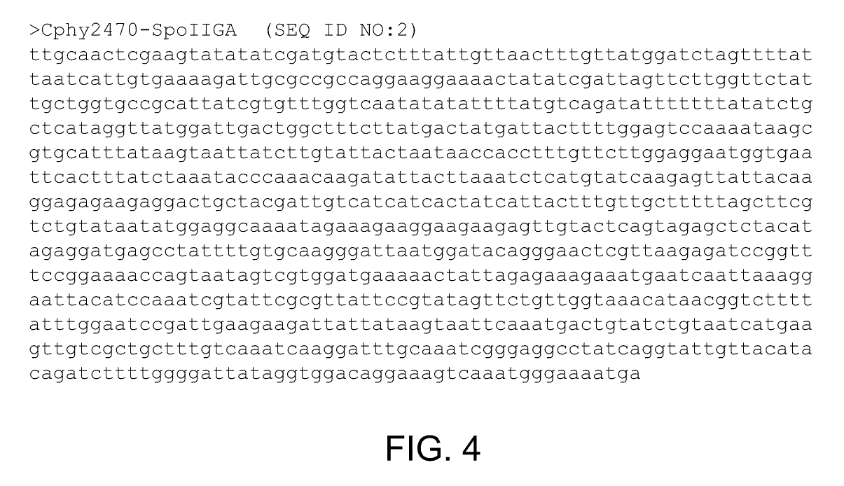

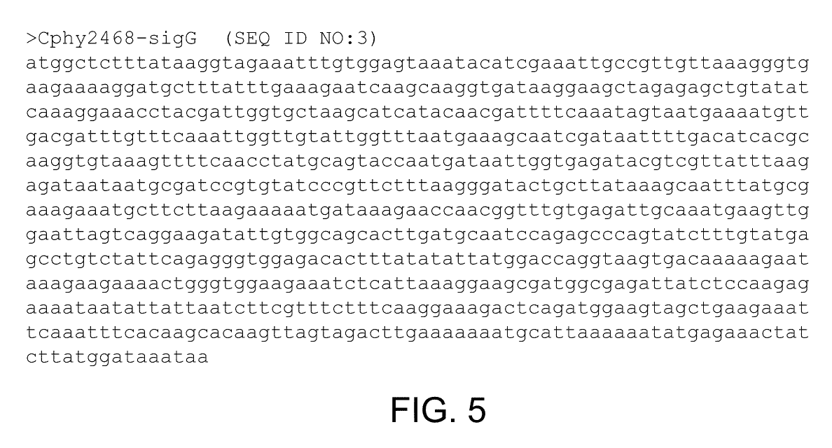

The Cphy2497 sequence was identified using BLAST (Basic Local Alignment Search Tool; Altschul, S. F., et al., (1993) J. Mol. Biol. 215:403-410; see also www.ncbi.nlm.nih.gov/BLAST/), by searching in the BLAST non-redundant database limited to C. phytofermentans (taxid:66219) (comprising all non-redundant GenBank CDS translations, sequences derived from the 3-dimensional structure Brookhaven Protein Data Bank, the SWISS-PROT protein sequence database, EMBL, and DDBJ databases) for sequences with similarity to the phosphorylation-activated transcription factor Spo0A in C. acetobutylicum. Results showed the predicted protein sequence, Cphy2497, to have significant identity, namely an e-value=2×10⁻¹⁴, to Spo0A in C. phytofermentans GI 160880629 (FIG. 2).

### Example 2

### Transformation of C. phytofermentans with a Construct to Knock-Out Spo0A

The pIMP1 plasmid contains gram-positive and gram-negative origins of replication, and ampicillin and erythromycin resistance genes (Mermelstein, L. D., N. E. Welker, G. N. Bennett, and E. T. Papoutsakis. 1992. Expression of cloned homologous fermentative genes in C. acetobutylicum ATCC 824. Bio/Technology 10:190-195). The spo0A-ko plasmid is constructed to contain an inactive Spo0A gene from C. phytofermentans in place of the origin of replication for C. phytofermentans. C. phytofermentans is transformed by converting the cells to protoplasts prior to transformation.

(1) Protoplast production. Bacterial cells are preconditioned by growth to exponential phase (optical density of 0.8 at 660 nm) in GS2 medium (4 g/l KH₂PO₄; 6.5 g/l Na₂HPO₄; 2.1 g/l Urea; 2 g/l Cysteine HCl; 3 g/l sodium citrate; 6 g/l yeast extract; 1 ml/l resazurin, adjusted to pH 7 with KOH, cellobiose (0.3% w/v) and 100 ml/l salts (1 g MgCl₂.6H₂O; 0.15 g CaCl₂.2H₂O; 0.00125 g FeSO₄.7H₂O)) containing 0.4% (wt/vol) glycine. Glycine, when incorporated in place of alanine in the peptidoglycans composing the cell wall, changes the structural properties of the cell wall and weakens it. Cells are harvested by centrifugation at 12,000 g for 10 minutes. The supernatant is discarded and the pellet resuspended in 3 ml of buffer 1 (GS2 with 0.3 M Sucrose and 25 mM MgCl₂ and 25 mM CaCl₂). To remove the cell wall, lysozyme (1 mg/ml) and cells are incubated at 37° C. for 40 minutes. The protoplasts are then centrifuged at 2,600 g for 10 minutes. The supernatant is discarded and the pellet is washed gently with 3 ml of buffer 2 (GS2 with 0.3 M Sucrose). The protoplasts are pelleted by centrifugation at 2,600 g for 10 minutes. The supernatant is discarded and the pellet is resuspended in 0.5 ml of buffer 2.

(2) DNase inactivation. To inactivate the potential DNases, C. phytofermentans protoplasts are heat-treated at 55° C. for 15 minutes.

(3) Transformation. To prevent digestion from endogenous DNases, the plasmid is methylated prior to transformation. The spo0A-ko plasmid DNA (7.5 μg/ml), polyethylene glycol (PEG) 6000 [40% (wt/v)] and 0.5 ml of the C. phytofermentans protoplasts suspension can be mixed and incubated at 40° C. for 2 minutes. This mixture is then diluted with 5 ml of buffer 1 and incubated at 40° C. for 2 hours. Dilutions are added to liquid GS2 and plated on agar (2% w/v) GS2 plates supplemented with antibiotics ampicillin (100 μg/ml) and erythromycin (200 μg/ml). Plates are incubated at 30° C. anaerobically and checked for transformants after 4 to 6 days.

### Example 3

### Disruption of the SpoIIE Endogenous Gene in C. phytofermentans Strains

The SpoIIE gene of C. phytofermentans is disrupted by providing a C. phytofermentans cell with a vector designed to knock out the endogenous SpoIIE gene. The vector is transformed into C. phytofermentans by protoplast transformation. Transformants are selected for on selective plates. Recombinants where the endogenous SpoIIE gene has been disrupted are identified by genome analysis and PCR. The cells harboring a genomic disruption of the SpoIIE gene provide a complete inactivation of the SpoIIE protein, and ethanol production is dramatically increased using these strains.

### Example 4

### Construction of SpoIIE Antisense Vectors

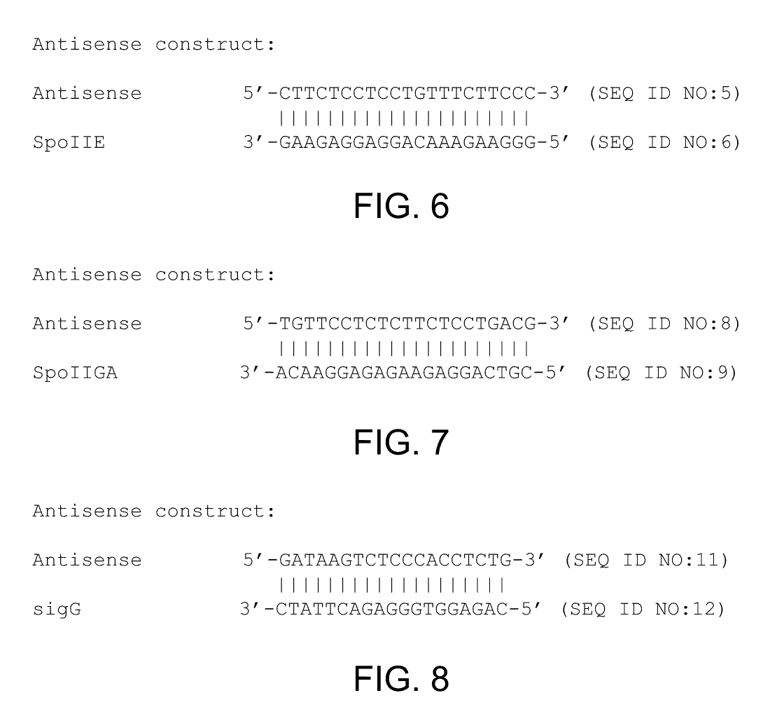

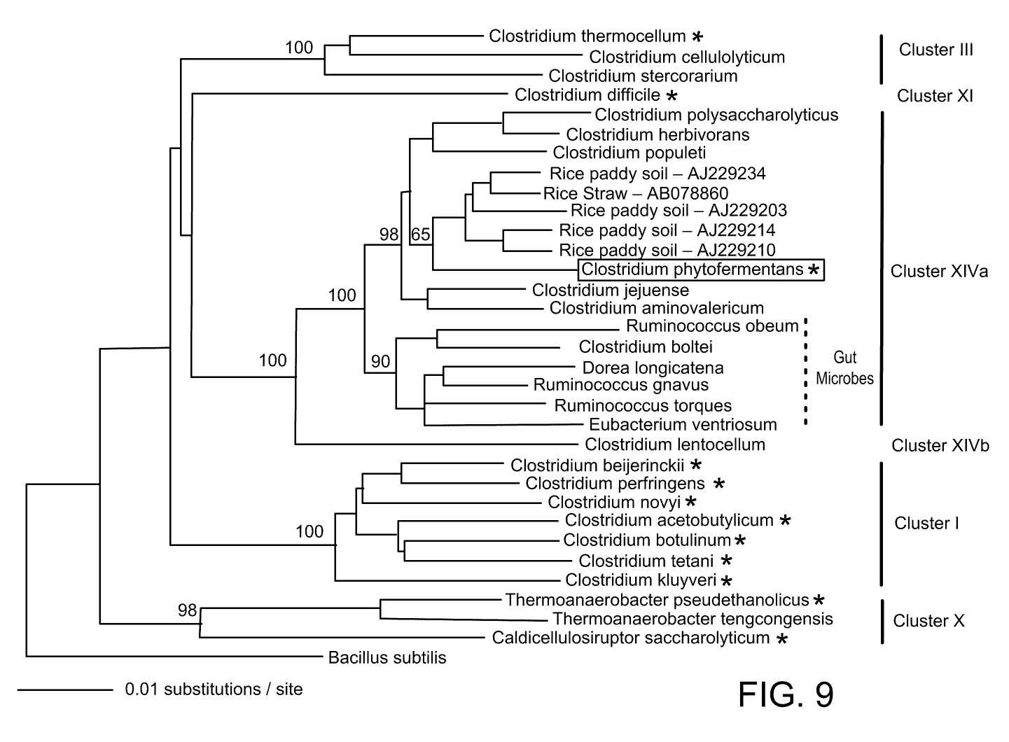

SpoIIE is downregulated by providing a C. phytofermentans cell with an antisense vector designed to reduce activity of the endogenous spoIIE gene. A spoIIE antisense construct was designed with the antisense design tool provided online at www.idtdna.com/Scitools/Applications/AntiSense/Antisense.aspx/Antisense.aspx using the SpoIIE gene sequence identified from C. phytofermentans (SEQ ID NO: 1). The predicted SpoIIE antisense sequence (SEQ ID NO: 4) “CCCTTCTTTGTCCTCCTCTTC” was used to design the SpoIIE complementary oligonucleotides that form the antisense construct shown in FIG. 6, namely, “SpoIIE-Top” “CTTCTCCTCCTGTTTCTTCCC” (SEQ ID NO: 5) and “SpoIIE-Bottom” “GGGAAGAAACAGGAGGAGAAG” (SEQ ID NO: 6).

The spoIIE complementary oligonucleotides are annealed together. Oligonucleotides “SpoIIE-Top” (SEQ ID NO: 5) and “SpoIIE-Bottom” (SEQ ID NO: 6) are diluted to a concentration of 0.5 μg/μl. 9 μl of the “SpoIIE-Top” and 9 μl of the “SpoIIE-Bottom” are mixed with 2 μl of 10×STE buffer (100 mM Tris-HCl, 500 mM NaCl, 10 mM EDTA, pH 8.0), and placed in a water bath set to 94° C. The water bath is allowed to cool to room temperature overnight, during which time the oligonucleotides anneal to form the antisense construct shown in FIG. 6.

The antisense construct is cloned into a suitable shuttle vector to create an SpoIIE antisense vector by techniques well known in the art. Suitable shuttle vectors can include, for example, plasmids with the ability to replicate in C. phytofermentans, and plasmids containing promoter sequences to express the antisense construct in C. phytofermentans.

The SpoIIE antisense vector is transformed into C. phytofermentans by protoplast transformation. Transformants are selected for on selective plates. Growth and product formation are determined in 120 hour fermentations of a strain where the SpoIIE antisense vector expresses the SpoIIE antisense sequence and a wild type control strain. Growth rates of both strains are measured using optical density at 600 nm. Acetone, butanol, and ethanol production are measured. It can be envisaged that by decreasing SpoIIE activity using an antisense oligonucleotide, the Clostridia will spend a greater amount of time undergoing ethanol production, and sporulation will be inhibited.

### Example 5

### Construction of SpoIIGA Antisense Vectors

SpoIIGA is downregulated by providing a C. phytofermentans cell with an antisense vector designed to reduce activity of the endogenous SpoIIGA gene. A SpoIIGA antisense construct was designed with the antisense design tool provided online at www.idtdna.com/Scitools/Applications/AntiSense/Antisense.aspx/Antisense.aspx using the SpoIIGA gene sequence identified from C. phytofermentans (SEQ ID NO: 2). The predicted SpoIIGA antisense sequence (SEQ ID NO: 7) “GCAGTCCTCTTCTCTCCTTGT” was used to design the SpoIIGA complementary oligonucleotides that form the antisense construct shown in FIG. 7, namely, “SpoIIGA-Top” “TGTTCCTCTCTTCTCCTGACG” (SEQ ID NO: 8) and “SpoIIGA-Bottom” “CGTCAGGAGAAGAGAGGAACA” (SEQ ID NO: 9).

The SpoIIGA complementary oligonucleotides are annealed together. Oligonucleotides “SpoIIGA-Top” (SEQ ID NO: 8) and “SpoIIGA-Bottom” (SEQ ID NO: 9) are diluted to a concentration of 0.5 μg/μl. 9 μl of the “SpoIIGA-Top” and 9 μl of the “SpoIIGA-Bottom” are mixed with 2 μl of 10×STE buffer (100 mM Tris-HCl, 500 mM NaCl, 10 mM EDTA, pH 8.0), and placed in a water bath set to 94° C. The water bath is allowed to cool to room temperature overnight, during which time the oligonucleotides anneal to form the antisense construct shown in FIG. 7.

The antisense construct is cloned into a suitable shuttle vector to create an SpoIIGA antisense vector by techniques well known in the art. Suitable shuttle vectors can include, for example, plasmids with the ability to replicate in C. phytofermentans, and plasmids containing promoter sequences to express the antisense construct in C. phytofermentans.

The SpoIIGA antisense vector is transformed into C. phytofermentans by protoplast transformation. Transformants are selected for on selective plates. Growth and product formation are determined in 120 hour fermentations of a strain where the SpoIIGA antisense vector expresses the SpoIIGA antisense sequence and a wild type control strain. Growth rates of both strains are measured using optical density at 600 nm. Acetone, butanol, and ethanol production are measured. It can be envisaged that by decreasing SpoIIGA activity using an antisense oligonucleotide, the Clostridia will spend a greater amount of time undergoing ethanol production, and sporulation will be inhibited.

### Example 6

### Construction of SigG Antisense Vectors

SigG is downregulated by providing a C. phytofermentans cell with an antisense vector designed to reduce activity of the endogenous SigG gene. A SigG antisense construct was designed with the antisense design tool provided online at www.idtdna.com/Scitools/Applications/AntiSense/Antisense.aspx/Antisense.aspx using the SigG gene sequence identified from C. phytofermentans (SEQ ID NO: 3). The predicted SigG antisense sequence (SEQ ID NO: 10) “GTCTCCACCCTCTGAATAG” was used to design the SigG complementary oligonucleotides that form the antisense construct shown in FIG. 8, namely, “SigG-Top” “GATAAGTCTCCCACCTCTG” (SEQ ID NO: 11) and “SigG-Bottom” “CAGAGGTGGGAGACTTATC” (SEQ ID NO: 12).

The SigG complementary oligonucleotides are annealed together. Oligonucleotides “SigG-Top” (SEQ ID NO: 11) and “SigG-Bottom” (SEQ ID NO: 12) are diluted to a concentration of 0.5 μg/μl. 9 μl of the “SigG-Top” and 9 μl of the “SigG-Bottom” are mixed with 2 μl of 10×STE buffer (100 mM Tris-HCl, 500 mM NaCl, 10 mM EDTA, pH 8.0), and placed in a water bath set to 94° C. The water bath is allowed to cool to room temperature overnight, during which time the oligonucleotides anneal to form the antisense construct shown in FIG. 8.

The antisense construct is cloned into a suitable shuttle vector to create an SigG antisense vector by techniques well known in the art. Suitable shuttle vectors can include, for example, plasmids with the ability to replicate in C. phytofermentans, and plasmids containing promoter sequences to express the antisense construct in C. phytofermentans.

The SigG antisense vector is transformed into C. phytofermentans by protoplast transformation. Transformants are selected for on selective plates. Growth and product formation are determined in 120 hour fermentations of a strain where the SigG antisense vector expresses the SigG antisense sequence and a wild type control strain. Growth rates of both strains are measured using optical density at 600 nm. Acetone, butanol, and ethanol production are measured. It can be envisaged that by decreasing SigG activity using an antisense oligonucleotide, the Clostridia will spend a greater amount of time undergoing ethanol production, and sporulation will be inhibited.

### Other Embodiments

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention as embodied in the attached claims.

What is claimed is:

1. A method for producing a compound, the method comprising:
incubating a culture medium with a Clostridium phytofermentans cell, wherein said cell has altered activity of at least one gene associated with sporulation as compared to the activity of the gene in a wild-type C. phytofermentans strain, and wherein said altered activity enhances production of said compound; and
isolating the compound from the culture medium.

2. The method of claim 1, wherein said compound is ethanol.

3. The method of claim 1, wherein said compound comprises at least one solvent selected from the group consisting of propanol, proprionate, acetate, lactate, and formate.

4. The method of claim 1, wherein said gene associated with sporulation is a gene upregulated by Spo0A.

5. The method of claim 1, wherein said gene associated with sporulation is selected from the group consisting of SpoIIAA, SpoIIAB, SigF, SpoIIE, SpoIIGA, SigG, and SigE.

6. The method of claim 1, wherein said gene associated with sporulation is SpoIIE.

7. The method of claim 6, wherein said altered activity is reduced activity of said gene.

8. The method of claim 1, wherein said altered activity is obtained by introducing into the cell an oligonucleotide that targets the gene.

9. The method of claim 8, wherein said oligonucleotide is introduced into the cell by expression from a plasmid that has been introduced into the cell.

10. The method of claim 1, wherein said altered activity is obtained by deleting or mutating at least a portion of the gene associated with sporulation.

11. The method of claim 1, wherein the culture medium comprises a biomass material.

12. The method of claim 11, wherein the biomass material comprises a plant polysaccharide.

13. The method of claim 11, wherein the biomass material comprises a lignocellulosic material.

14. An isolated Clostridium phytofermentans cell comprising altered sporulation activity in comparison to a wild-type strain of C. phytofermentans.

15. The cell of claim 14, comprising an alteration of at least one gene associated with sporulation.

16. The cell of claim 14, wherein said gene is selected from the group consisting of SpoIIAA, SpoIIAB, SigF, SpoIIE, SpoIIGA, SigG, and SigE.

17. The cell of claim 15, wherein said gene is upregulated by Spo0A.

18. The cell of claim 15, wherein said gene is SpoIIE.

19. The cell of claim 15, wherein said alteration comprises disruption of gene activity by an oligonucleotide.

20. The cell of claim 15, wherein said alteration comprises at least a partial deletion or mutation.

21. The cell of claim 19, further comprising an expression plasmid encoding said oligonucleotide.

22. A method for reducing sporulation in a Clostridium phytofermentans cell, the method comprising reducing the activity of a gene associated with sporulation.

23. The method of claim 22, wherein reducing comprises disrupting the gene.

24. The method of claim 22, wherein said reducing the activity comprises providing an oligonucleotide to the strain.

25. The method of claim 24, wherein the oligonucleotide is provided by an expression plasmid.

26. The method of claim 22, wherein reducing the activity comprises deleting or mutating at least a portion of the gene.

27. A fuel producing mixture comprising:
an isolated Clostridium phytofermentans microorganism comprising an alteration of at least one gene associated with sporulation; and
biomass.

28. The mixture of claim 27, wherein the gene is selected from the group consisting of SpoIIAA, SpoIIAB, SigF, SpoIIE, SpoIIGA, SigG, and SigE.

29. The mixture of claim 27, wherein the gene is upregulated by Spo0A.

30. The mixture of claim 27, wherein the gene is SpoIIE.

31. The mixture of claim 27, further comprising a second microorganism.

32. The mixture of claim 31, wherein the second microorganism is a yeast, fungus, or bacterium.
